(19) United States
(12) Patent Application Publication
(10) Pub. No.: US 2002/0160053 A1
(43) Pub. Date: Oct. 31, 2002

(54) Solution for promoting growth of tissue cells at wound sites and production process therefor

(76) Inventors: Naoki Yahagi, Shiga (JP); Osao Sumita, Tokyo (JP)

Correspondence Address: PILLSBURY WINTHROP, LLP, P.O. BOX 10500, MCLEAN, VA, 22102, US

(21) Appl. No.: 10/146,140

(22) Filed: May 16, 2002

Related U.S. Application Data
(63) Continuation-in-part of application No. 09/714,826, filed on Nov. 17, 2000.

(30) Foreign Application Priority Data
Nov. 17, 1999 (JP) 326993/1999

(51) Int. Cl. A61K 33/00; A61K 33/14; C25C 1/02

(52) U.S. Cl. 424/600; 424/661; 205/620

(57) ABSTRACT

A tissue cell growth-promoting solution produced by this invention comprising water containing at least 1 to 500 ppm of active oxygen, when applied to a wound, supplies active oxygen originating from outside the biobody to supplement the active oxygen produced by the biobody's own protective system cells such as neutrophils and macrophages which gather at the wound site, thus increasing the concentration of active oxygen at the site of the wound, mimicking a state in which a large quantity of such bio-signals is secreted by the biobody itself, to promote the reconstruction of tissues, the action corresponding to the last of the four main steps involved in wound healing biochemical processes of “blood vessel reaction”, “blood vessel coagulation”, “inflammation”, “reconstruction of tissues” and which would otherwise have to rely on the natural healing power of the biobody itself.

This is a Continuation-In-Part of and claims priority to US non-provisional application Ser. No. 09/714,826 filed Nov. 17, 2000 and claims priority to Japanese Patent Application No. 326,993/1999 filed Nov. 17, 1999. The contents of each of these applications are hereby incorporated by reference herein in their entireties.

### BACKGROUND OF THE INVENTION

1. Field of the Invention

This invention concerns the making of a solution for promoting the growth of tissue cells at the site of a wound, a process that promotes regeneration of tissue and wound healing.

2. Related Art Statement

Wound means a pathological state in which tissue either inside or on the outer surface of a biobody is fragmented or damaged, with accompanying less or restriction of the functions of the affected tissues.

It has long been known that when a wound is inflicted, the affected region heals through four steps: “blood vessel reaction”, “blood clotting”, “inflammation”, and “reconstruction of tissue”.

Specifically, when a biobody's tissues are damaged so as to cause bleeding, the blood vessel reactions that occur lead first to the formation of clots together with constriction of blood vessels, and this is followed by dilation of the blood vessels after the bleeding stops.

This dilation increases the blood flow at the periphery of the wound, and blood cells and plasma are transferred to, and coagulated at the wound site to cause further clotting, which temporarily closes the wound.

Further, immunoprotective cells gather at the periphery of a wound when the blood flow has increased; to activation of cell-mediated or humoral immunoprotective reactions which cause inflammation.

Finally, the tissues of blood vessels themselves are reconstructed by the action of transmission factors or growth factors released within the wound region, and growth factors secreted by platelets or macrophages provide stimulation to increase the movement of fibroblasts so as to promote reconstruction of connective tissues and, further, form granulations.

In such situation, it is now known that active oxygen produced from bioprotective cells (such as neutrophils or macrophages) that have gathered at the wound site act as bio-signals and activate various enzymes and factors to promote the reconstruction of the tissues. That is, a necessary condition for tissue reconstruction is that the relevant enzymes or factors are activated, for which it is necessary that active oxygen is produced in the region of the wound.

It should be mentioned here that drugs used for promoting wound healing are generally hemostatic for suppressing bleeding in the wound region, anti-inflammatory for suppressing inflammation, sterilizers for sterilization so as to prevent miscellaneous bacteria from invading the wound, or drugs having more than one of the above pharmaceutical effects. However, at present “reconstruction of tissue” as the final stage of wound healing has to rely on the auto-therapy inherent in living bodies.

Grady (U.S. Pat. No. 5,084,011) and Kolta (U.S. Pat. No. 6,139,876) disclose techniques of healing wounds by providing an atmosphere containing dissolved oxygen at a high concentration for wound sites by utilizing a gel or the like containing oxygen at high concentration.

Individual biobody cells conduct metabolism of intaking oxygen and discharging carbon dioxide, so that metabolism is conducted vigorously when the biobody cells are in an atmosphere at a high concentration of dissolved oxygen.

Accordingly, it is well-known that when biobody cells no more conducting vigorous metabolism because of wounds, etc. are put in an atmosphere of oxygen at high concentration, the metabolism is promoted more than in a case of a lower concentration, to activate the cells thereby healing diseases or wounds. In view of the above, Grady and Kolta discloses means for positively supplying oxygen consumed in the metabolism at a high concentration to the biobody cells. That is, Grady and Kolta intend to supply oxygen to be consumed in the metabolism of biobody cells in a state capable of conducting metabolism positively or in a great amount.

When wounds damage biobody (living body) tissues, it would be insufficient to merely activate existent cells but biobody (living body) tissues have to be regenerated (reconstructed) by growing cells to form new biobody cells.

However, when oxygen is supplied as in the invention of Grady or Kolta, while the biobody cells present at the periphery of wound sites can be activated but growth of new biobody cells has to rely on the auto-therapy of living bodies.

This is because the metabolism and the growth of the biobody cells are processes quite different from each other, and oxygen is consumed only in the metabolism but does not directly contribute to the growth of the biobody cells.

Then, in the auto-therapy, the reconstruction of the biobody tissues at the wound site is conducted through the following processes:

(a) Macrophages gathering at the wound site yield growth factors and enzymes,

(b) The growth factors and the enzymes are activated,

(c) The activated growth factors and enzymes give stimulations to increase or movement of fibroblasts to grow the biobody cells, and

(d) The tissue is reconstructed (regenerated) by the grown new biobody cells.

In view of the above, it is a technical object of this invention to provide a chemical solution for promoting the growth of tissue cells at wound sites, specifically to aid wound healing by promoting the reconstruction of tissues by enhancing certain biochemical reactions in and around the region of the wound, by the process similar with that of auto-therapy of promoting the growth of biobody cells by activation of the growth factor and enzymes.

### SUMMARY OF THE INVENTION

The present invention allows the aforementioned object to be achieved by producing an aqueous solution for promoting the growth of tissue cells at wound sites. This solution comprises water containing at least 1 to 500 ppm of active oxygen and 10 to 10000 ppm of halogen ions. The active oxygen can include singlet oxygen (¹O₂) formed by excitation of triplet oxygen, superoxide (O₂⁻) formed by reduction of oxygen by a single electron, and hydroxy radical (HO.), as well as hypochlorous ions (ClO⁻) and peroxy radical (ROO.), alkoxy radical (RO.), and hydroperoxide (ROOH) and formed by reactions with biobody ingredients such as unsaturated aliphatic acids (R).

In the case of auto-therapy, macrophages gathering at the wound sites produce active oxygen and the active oxygen functions as bio-signals to activate the growth factors and the enzymes yielded also from the macrophage.

That is, it is known that active oxygen contributes to activation of growth factors and enzymes in process of auto-therapy. Specifically, in this invention, active oxygen supplied from the outside activates the growth factors and the enzyme together with active oxygen produced spontaneously from macrophage and promotes the growth of the biobody cells by the process similar with that of the auto-therapy.

In this case, since the amount of active oxygen is greater by so much as it is supplied from the outside than the naturally produced amount to increase the concentration of the active oxygen in the would site, this provides a state mimicking to a case where a greater amount of bio-signals are secreted.

As a result, growth of the biobody cells is further promoted and the tissues at the wound site are reconstructed (regenerated) in a short period of time, so that this can provide an effect that the healing for the wounds can also be promoted.

The active oxygen is quite different in view of chemical species and biochemical activities from ordinary molecular oxygen.

While ordinary oxygen contributes only to metabolism but does not function as bio-signals for the growth of the cells, the active oxygen does not contribute to the metabolism but function as bio-signals for the growth of the cells.

When the active oxygen is less than 1 ppm, it does not function as bio-signals since it is much smaller compared with the amount produced in the auto-therapy for wound sites. On the other hand, when the active oxygen is 500 ppm or more, it is not suitable as a solution for promoting the growth of cells since the toxicity of the active oxygen gives undesired effects on the biobody cells.

The solution produced according to this invention preferably comprises water containing not only active oxygen, but also halogen ions as a prime ingredient. On the basis of experiments conducted by the present applicants, it is considered that the solution is more similar to the body's own fluids when halogen ions are present, since halogen ions are a constituent of the fluid formed by tissue cells, and since the halogen ions and the active oxygen contained in this solution function effectively as bio-signals.

In this invention, it is preferred that the active oxygen is hydroxy radical HO. since the oxygen produced in the biobody is hydroxy radical HO..

However, since the hydroxy radical HO. can not be synthesized directly it is practical and preferred in this invention to use a precursor for the hydroxy radical HO. that forms hydroxy radical in the presence of an iron or copper ingredient as a catalyst, for example, hypochlorous ion ClO⁻. When the precursor is supplied to a wound site, it reacts with the iron ingredient in blood to produce the hydroxy radical HO..

In this invention it is preferred to adjust the pH of the solution for promoting the growth of tissue cells at the wound site to between 6 and 8, which is approximate to the pH 7.4 of body fluids.

Further, it is preferred in this invention that sodium chloride (NaCl) is added to the solution until the solution is isotonic with body fluids (i.e., of a similar osmotic pressure). Thus the solution has ingredients in common with the body fluids, as well as a similar pH and osmotic pressure, and it is considered that, as a consequence, the active oxygen contained therein functions more effectively as a bio-signal. Furthermore, it has been experimentally confirmed that the solution according to this invention at a pH similar to that of the body fluids is effective for internal wounds, as well as for wounds at the body's external surface.

This invention also provides a process for producing a solution for promoting the growth of tissue cells, which can produce the solution simply at a reduced cost such as even in hospitals.

### DESCRIPTION OF THE ACCOMPANYING DRAWINGS

FIG. 1 is an explanatory view illustrating a two-chamber type electrolysis apparatus that can be used to manufacture a solution for promoting the growth of tissue cells at wound sites.

FIG. 2 is an explanatory view illustrating a second type of electrolysis apparatus.

FIG. 3 is an explanatory view illustrating the three-chamber type of electrolysis apparatus.

FIG. 4 is a graph showing absorption spectra for anode-electrolyzed water produced according to this invention, as well as for chlorous acid and hypochlorous acid.

FIG. 5 is a graph showing ESR (electron-spin resonance) spectra for anode-electrolyzed water produced according to invention.

FIG. 6 is a table showing effects of various treatments on the recovery of skin wounds with time.

FIG. 7 is a table showing the characteristics of the solutions according to this invention and of other types of water or solution.

FIG. 8 is a table showing effects of various treatments on the recovery of internal wounds with time.

### DESCRIPTION OF PREFERRED EMBODIMENTS

This invention is now explained more fully and concretely by describing its preferred embodiments with reference to the accompanying drawings.

The invention employs chemicals and the electrolysis of water to form active oxygen for use in a solution for promoting the growth of tissue cells at wound sites. The use of chemicals involves the use of hydrogen peroxide to OH. radicals by Fenton reaction or the use of ozone. Ozone itself is not active oxygen, but active oxygen is formed in the course of its decomposition.

The active oxygen formed in biobody cells has bio-signaling functions. Hence, for active oxygen supplied from the outside to a wound site to function as an effective agent for the promotion of the growth of tissue cells, it is desirable that the solution within which it is contained should approximate as closely as possible to the body fluids composition, pH, and osmotic pressure.

For this purpose, in the solution in this example, halogen ions typically represented by chlorine ions and oxygen series oxidative substance such as ozone are used in combination.

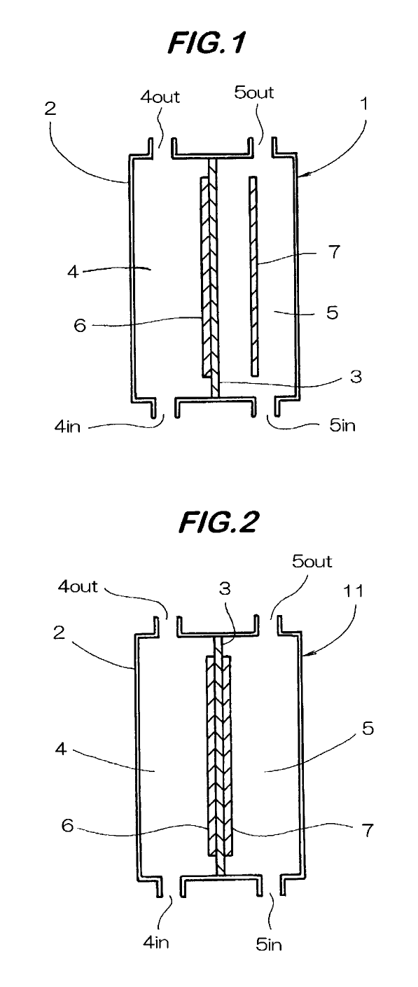

The electrolysis apparatuses 1,11, and 12 (see FIGS. 1, 2, and 3) can each form such a mixture easily. In FIGS. 1, 2, and 3, the same or similar parts carry the same reference number in each figure. A detailed explanation of the various parts will be given later.

In the two-chamber type electrolysis apparatus 1 shown in FIG. 1, electrolysis vessel 2 is partitioned by fluoric cation exchange membrane 3, acting as a diaphragm, into anode chamber 4 and cathode chamber 5. Anode electrode 6 is in intimate contact with cation exchange membrane 3, while cathode electrode 7 is located within cathode chamber 5. In anode chamber 4 and cathode chamber 5, are provided inlets 4in and 5in, respectively, and outlets 4out and 5out, respectively. Platinum-plated titanium is used for each of electrodes 6 and 7. Purified water is supplied to anode chamber 4 and saline formed by dissolving sodium chloride in purified water is supplied to cathode chamber 5.

In the second two-chamber type electrolysis apparatus (11, shown in FIG. 2), both anode electrode 6 and cathode electrode 7 may be brought into closely contact with cation exchange membrane 3.

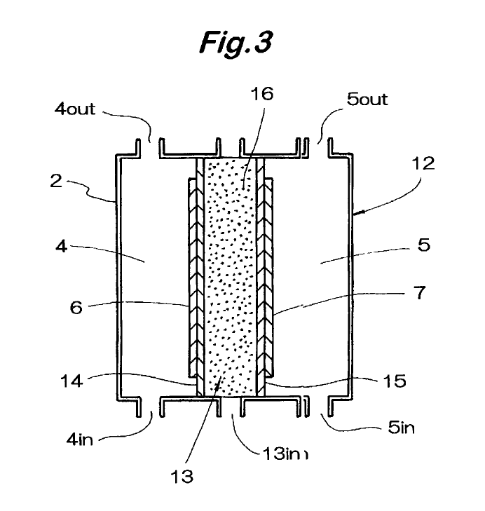

A three-chamber type electrolysis apparatus 12 may also be used as shown in FIG. 3 in which the middle chamber 13 is located between anode chamber 4 and cathode chamber 5. Using a pair of diaphragms 14 and 15 partitions chambers 4, 5, and 13. The diaphragms 14 has a composite diaphragm structure comprising Nafion 117 (trade name of product manufactured by DuPont Co.) acting as a fluoric cation-exchange membrane, and AMV (trade name of product manufactured by Tokuyama Soda Co.) acting as an anion-exchange membrane, stacked to each other. The diaphragm 15 is made of cation-exchange membrane Nafion 117 as described before.

Each of the diaphragms is attached with the cation-exchange membrane being faced to anode chamber 4, and anode electrode 6 and cathode electrode 7 are placed in closely contact with these diaphragms 14 and 15, respectively as shown in FIG. 3. Further, granular cation-exchange resin 16 fills middle chamber 13.

When the three-chamber type electrolysis apparatus 12 is used, saline is supplied to middle chamber 13 via inlet 13in, while purified water is supplied to anode chamber 4 and cathode chamber 5 via inlets 4in and 5in, respectively.

When electrolysis is conducted by using electrolysis apparatus 1, 11, or 12 shown in FIGS. 1, 2, and 3, respectively, sufficient chlorine ions are not supplied to the surface of anode electrode 6, so an oxidative decomposing reaction occurs as shown below.

2H₂O+O₂-4e⁻→4H⁺+2O₃

2H₂O-3e⁻→3H⁺+HO₂

2H₂O-2e⁻→2H⁺+H₂O₂

As described above, the anode-electrolyzed water formed in the anode chamber contains ozone, active oxygen, and hydrogen peroxide.

Further, since a proportion of the chorine ions present moves from cathode chamber 5 or middle chamber 13 into anode chamber 14, oxidative substances containing oxygen and chlorine ions are necessarily present together.

In an aqueous solution in which oxidative substances containing oxygen and halogen ions are present together, complex compounds are formed transiently between such various substances. For instance, when ozone and chlorine ions are present together, they do not react instantly to form hypochlorous ions as a precursor for the hydroxy radical but rather form quasi-stable complexes capable of forming hypochlorous ions and then form the hypochlorous ions by the subsequent reaction.

It is considered that the hypochlorous ions described above have biological effects in the biobody and react with proteins or amino acids to form hydroxy radicals and the latter promotes, by bio-signaling, the growth of tissue cells.

Since oxidative active species formed from ozone or by electrolysis have inherent antibacterial actions, they function both to promote the growth of tissue cells and to sterilize the wound sites during wound healing.

Then the three-chamber type electrolysis apparatus 12 shown in FIG. 3 is used, and purified water is supplied at a flow rate of 0.5 l/min to anode chamber 4 and also to cathode chamber 5, an aqueous solution containing 10 to 10000 ppm of chlorine ions Cl⁻¹ dissolved in the purified water is supplied at a flow rate of 0.2 l/min to middle chamber 13.

The area of each of electrodes 6 and 7 is 48 cm², and the electrolysis current is 5 A.

For the cathode-electrolyzed water, the pH is about 11.5 and ORP is −850 mV, while for the anode electrolyzed water, the pH is about 2.4 and ORP is −1150 mV.

Then, a slight amount of hydroxy radicals HO., as well as oxidative substances such as ozone are formed at the anode electrode. The oxidative substances such as ozone react with chlorine ions that move from middle chamber 13 to anode chamber 4 to form hypochlorous ions as the precursor for the hydroxy radical.

Consequently, anode-electrolyzed water containing a slight amount of hydroxy radicals HO., hypochlorous ions ClO⁻ as the precursor for the hydroxy radicals and chlorine ions (halogen ions) dissolved therein is obtained.

However, it is considered, in view of the absorption spectra, that the reaction takes place gradually in which hypochlorous acid is not formed directly but the precursor for the hypocilorous acid is formed.

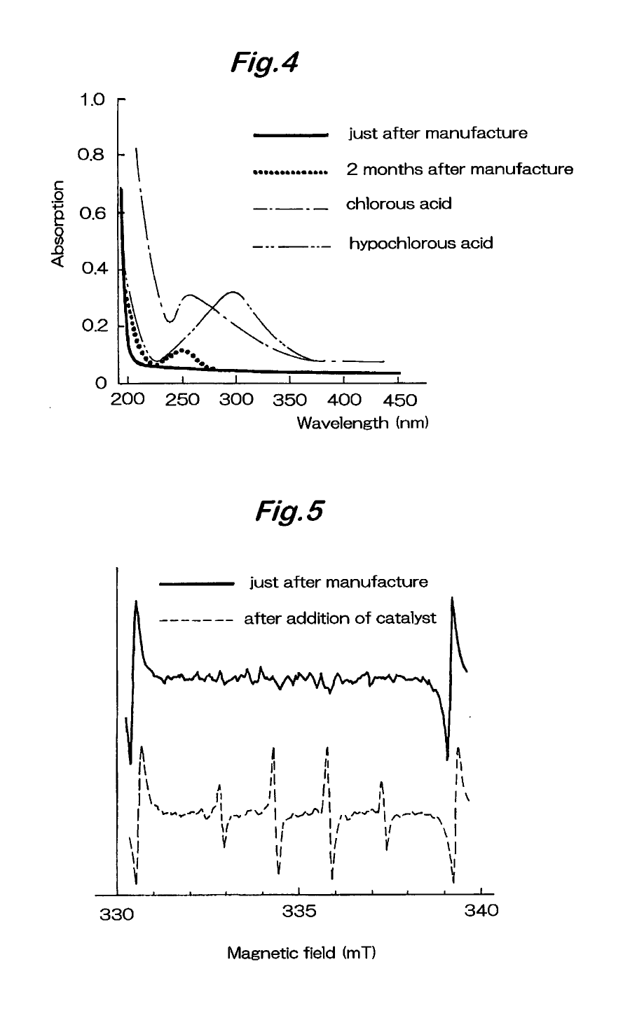

FIG. 4 is the graph showing absorption spectra for the anode-electrolyzed water thus formed just after formation and after a period of time. According to this graph, since absorption in the near-ultraviolet representing hypochlorous acid or chlorous acid is not observed just after formation, it is apparent that hypochlorous acid and chlorous acid are not formed. Since absorption in the near-ultra violet region is observed with the lapse of the reaction time, it can be seen that hypochlorous acid and chlorous acid are formed.

FIG. 5 is a graph showing ESR spectra (Electron Spin Resonance Spectra) obtained before and after adding DMPO (5,5-dimethyl-1-pyrroline-5-oxide) as a radical scavenger to the anode-electrolyzed water thus formed. It can be seen from the graph that although no significant absorption spectrum is observed for the anode-electrolyzed water before the addition of iron ions, four absorption lines representing OH radicals are present after its addition. Thus, active oxygen is formed in the presence of a catalyst such as iron.

Experiment 1

Anode-electrolyzed water with 1 to 500 ppm concentration of active oxygen, such as hydroxy radicals HO. and hypochlorous ions ClO⁻ as the precursor therefor and at 10 to 10,000 ppm concentration of halogen ions such as chlorine ions Cl⁻ is thus formed.

In the experiment, an anode-electrolyzed water with 80 to 130 ppm concentration of active oxygen, and with 1,000 to 4,000 ppm concentration of halogen ions such as chloride ions Cl⁻ is used, to which sodium chloride is added to make the concentration of chlorine ions to 5,500 ppm isotonic with that of physiological saline. Further, a solution L₁ for promoting the growth of tissue cells is formed by adding an aqueous solution of sodium hydroxide to adjust pH to 7.4 and a solution L₂ for promoting the growth of tissue cells at pH 2.4 as it is with no pH adjustment are formed. For comparison, physiological saline C₁ containing dissolved hypochlorous acid and cathode-electrolyzed water C₂ are used.

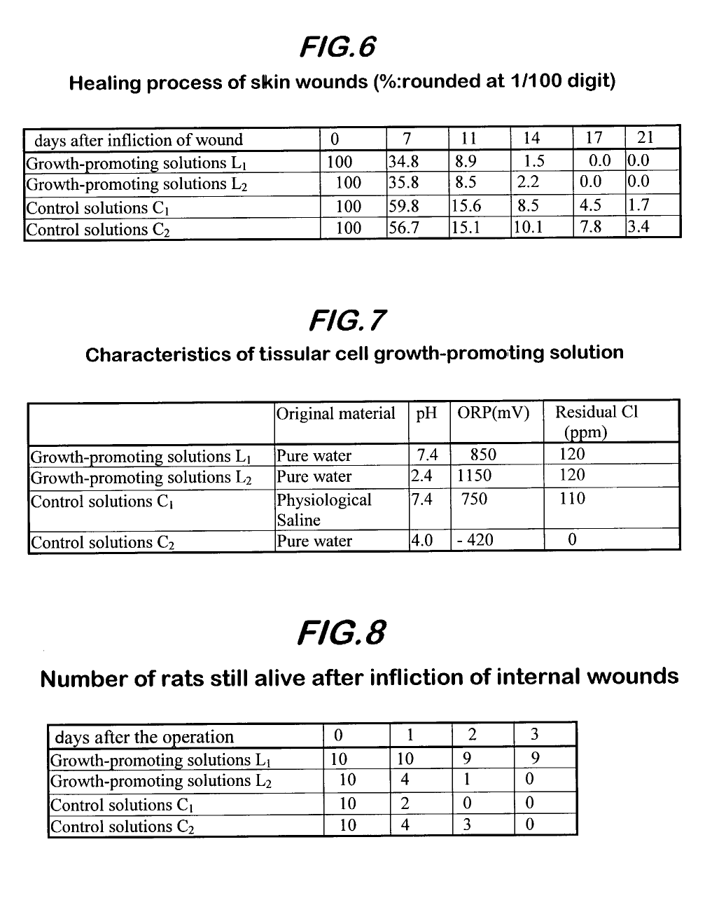

FIG. 6 shows the results of an experiment to examine recovery with time of skin wounds treated with the above solutions for confirming the effect of L₂, while FIG. 7 shows the characteristics of the solutions L₁ and L₂ for promoting the growth of tissue cells according to this invention and comparative solutions C₁ and C₂.

In the experiment, rats were used. The skin on their back was shaved, and then skins were cut each by 1 cm² to form wound sites.

Then, only for the first seven days, the putative solution for promoting the growth of tissue cells was applied dropwise twice per day while care being taken that the liquid did not overflow from the wound sites. Subsequently, the wound sites were left untouched. The area of the wound sites was determined by a planimetry method, and each was expressed as a percentage of its area on the first day.

The rats were then kept one per cage.

As shown by the table in FIG. 6, rats treated with each of the tissue cell growth-promoting solutions L₁ and L₂ showed complete healing of the treated wound after 17 days, whereas the rats treated with each of control solutions C₁ and C₂ did not show complete healing even after 21 days.

Further, when sodium chloride was added to make the solution isotonic with the body fluids, rats treated with the putative tissue cell growth-promoting solution L₁ or L₂ showed more rapidly wound healing than rats treated with control solution C₁ or C₂.

Using the 2-chamber type electrolysis apparatus 1 or 11 shown in FIG. 1 or FIG. 2, electrolysis is conducted by supplying purified water to anode chamber 4 and an aqueous solution containing 10 to 10,000 ppm of halogen ions is supplied to cathode chamber 5 to form an anode-electrolyzed water in anode chamber 4. The result of experiment for the tissue cell growth-promoting solution adjusted to pH to 7.4 with addition of sodium chloride to the thus formed anode-electrolyzed water was equivalent with that for the tissue cell growth-promoting solution L₁. —Experiment 2

In this experiment, a peritonitis model was prepared in rats, and the effect of the tissue cell growth-promoting solutions was confirmed. In each rat, the cecum was ligated at a position 3 to 5 mm from the ileum, and then punctured at two places at an intermediate position between the ligation and the distal end of the cecum and near the proximal end of the cecum using an 18 G injection needle. This was done deliberately to cause acute peritonitis.

A catheter was inserted from the back of the rat to pass beneath the skin and into the abdominal cavity. Then, one of the putative tissue cell growth-promoting solutions (L₁ or L₂) or one of the control solutions (C₁ or C₂) was injected 1 cc per 100 g of the body weight into the abdominal cavity so that it bathed the region expected to be afflicted by peritonitis.

The table in FIG. 8 shows the process of recovery observed in rats kept in cages for three days after the operation. According to these figures, which show the number of rats (out of 10 in each group) still alive on each day. The progress to peritonitis was not hindered except in the rats treated with the pH adjusted tissue cell growth-promoting solution Ll. Hence, it is considered that the pH has to be adjusted to neutral to achieve healing of internal wounds.

As described above, when the tissue cell growth-promoting solution according to this invention is applied to a wound site, the active oxygen from outside the biobody supplements active oxygen produced from the body's bioprotective cells such as neutrophils or macrophages gathered at the site of the wound. As a consequence, the concentration of active oxygen at the wound site is increased, mimicking a state in which a large quantity of bio-signals is secreted by the biobody itself In this way, a solution according to this invention can provide an excellent enhancement of the reconstruction of tissue cells that would occur naturally. As a result, wound-healing takes place much more quickly.

What is claimed is:

1. A solution for promoting the gloss of tissue cells at the site of a wound, comprising water containing at least 1 to 500 ppm of active oxygen and 10 to 10,000 ppm of halogen ions.

2. A solution for promoting the gloss of tissue cells at the site of a wound according to claim 1, wherein the halogen ion is chlorine ion.

3. A solution for promoting the gloss of tissue cells at the site of a wound according to claim 1, wherein the active oxygen comprises hydroxy radicals (HO.).

4. A solution for promoting the gloss of tissue cells at the site of a wound according to claim 1, wherein a hydroxy radical precursor for forming hydroxy radical (HO.) in the presence of iron or copper ions as a catalyst is used for forming active oxygen.

5. A solution for promoting the gloss of tissue cells at the site of a wound according to claim 1, wherein the pH is between 6 and 8.

6. A solution for promoting the gloss of the tissue cells at the site of a wound according to claim 1, wherein sodium chloride is added to make the solution isotonic with body fluids.

7. A process for producing a solution for promoting the growth of tissue cells at the site of a wound by electrolysis using a an electrolysis apparatus which is partitioned into an anode chamber and, a cathode chamber by way of diaphragms, by supplying water to the anode chamber and supplying an aqueous solution containing 10 to 10,000 ppm of halogen ions to the cathode chamber, thereby taking out the anode-electrolyzed water formed in the anode chamber as a solution for promoting the growth of tissue cells at the site of a wound.

8. A process for producing a solution for promoting the growth of tissue cells at the site of a wound by electrolysis using a thee-chamber type electrolysis apparatus which is partitioned into an anode chamber, a cathode chamber and an middle chamber situated therebetween by way of a pair of diaphragms, a cation exchange membrane is used as the diaphragm for partitioning the anode chamber and the middle chamber and the anode electrode is in closely contact with the cation exchange membrane, by supplying water to the anode chamber and the cathode chamber and supplying water containing 10 to 10,000 ppm of halogen ions to the middle chamber, thereby taking out the anode-electrolyzed water formed in the anode chamber as a solution for promoting the growth of tissue cells at the site of a wound.

9. A process for producing a solution for promoting the growth of tissue cells at the site of a wound according to claim 8, wherein the cation exchange membrane is a fluorinated cation exchange membrane.

10. A process for producing a solution for promoting the growth of tissue cells at the site of a wound according to claim 8, wherein the diaphragm for partitioning the anode chamber and the middle chamber comprises a composite diaphragm structure having a fluorinated cation exchange membrane and an anion exchange membrane stacked to each other, and the anode electrode is in closely contact with the fluorinated cation exchange membrane.
